(19) United States
(12) Patent Application Publication
(10) Pub. No.: US 2026/0165843 A1
(43) Pub. Date: Jun. 18, 2026

(54) PROSTHETIC TIBIAL COMPONENT HAVING PIVOTABLE, SHOCK-ABSORBING ARTICULAR PADS

(71) Applicants: Thomas A. Bunner, Fort Wayne, IN (US); William J. Berghoff, Fort Wayne, IN (US)

(72) Inventors: Thomas A. Bunner, Fort Wayne, IN (US); William J. Berghoff, Fort Wayne, IN (US)

(21) Appl. No.: 18/980,071

(22) Filed: Dec. 13, 2024

(51) Int. Cl. A61F 2/38

(52) U.S. Cl. CPC A61F 2/389; A61F 2/3868; A61F 2210/0004; A61F 2210/0014

(57) ABSTRACT

A tibial component of a prosthetic knee implant comprises an implant tray, medial and lateral articular pads, first and second pivot bearings, and first and second cushion members. The implant tray includes medial and lateral sockets. The medial articular pad is supported within the medial socket by the first pivot bearing which engages the socket's bottom surface and the first cushion member which is sandwiched between the first pivot bearing and the medial articular pad. The lateral articular pad is supported within the lateral socket by the second pivot bearing which engages the socket's bottom surface and the second cushion member which is sandwiched between the second pivot bearing and the lateral articular pad. The pivot bearings pivot, roll, and slide against the socket bottom surfaces whereby the articular pads can pivot and rotate within the sockets. The cushion members absorb forces and impacts experienced by the articular pads.

### BACKGROUND OF THE INVENTION

### 1. Field of the Invention

The present invention relates to prosthetic components used for knee replacement surgeries. More particularly, the present invention is directed to a prosthetic tibial component which replicates the articular structures and functions of a natural tibial plateau and includes pivotable, shock-absorbing articular pads.

### 2. Background

The knee joint is the largest joint in the human body and comprises the interface between the flat, shelf-like upper portion of the tibia, referred to as the tibial plateau, and the oblong, knuckle-shaped lower portions of the femur, referred to as the femoral condyles. The tibial plateau comprises a pair of concave articular surfaces, referred to as the tibial condyles, and a pair of C-shaped cartilage structures called menisci which are supported above the tibial condyles. As the knee joint flexes/bends and extends/straightens, the femoral condyles roll/pivot/slide on the tibial condyles. The C-shaped menisci, which are sandwiched between the tibial and femoral condyles, act as cushions which absorb shocks and impacts experienced by the knee joint.

Typically, a healthy knee joint can extend until the leg is completely straight and can flex/bend until the calf touches the back of the thigh. However, injuries and disease can damage or degrade the tibial plateau and/or the femoral condyles which can limit the range of motion of the joint and often results in pain or discomfort. In severe cases, a total knee arthroscopy (TKA) or other surgical procedures may be necessary to restore mobility to the injured or diseased knee joint. In a TKA procedure, the damaged portions of the knee joint are surgically removed or “resected” and are replaced with the components of a prosthetic knee implant.

Traditionally, prosthetic knee implants comprise a femoral component which secures to the lower end of the patient's femur and a tibial component which secures to the upper end of the patient's tibia. The femoral component is shaped to replicate the lower end of the femur and includes a pair of oblong, knuckle-shaped artificial femoral condyles. The tibial component typically comprises an implant tray which is secured to the patient's tibia and one or more contact pads which are rigidly secured to the implant tray and are shaped to mimic the tibial condyles. In use, the artificial femoral condyles engage and roll/pivot/slide on the contact pad(s) as the knee joint flexes and extends, thereby replicating the function of a natural knee joint.

Traditional knee implants, however, are known to limit the range of motion of the knee joint and typically do not include shock absorbing components like the cartilage and menisci of a natural knee joint. Accordingly, a need exists for an improved prosthetic knee implant.

### SUMMARY OF THE INVENTION

In the present invention, a tibial component of a prosthetic knee implant can comprise an implant tray having a medial tray socket and a lateral tray socket, a medial articular pad that is sized and shaped to fit into the medial tray socket, a lateral articular pad that is sized and shaped to fit into the lateral tray socket, a first and a second pivot bearing, and a first and a second cushion member. The medial articular pad is supported within the medial tray socket by the first pivot bearing which engages the bottom surface of the medial tray socket and the first cushion member which is sandwiched between the first pivot bearing and the medial articular pad. The lateral articular pad is supported within the lateral tray socket by the second pivot bearing which engages the bottom surface of the lateral tray socket and the second cushion member which is sandwiched between the second pivot bearing and the lateral articular pad. In use, the first and second pivot bearings pivot, roll, and slide against the socket bottom surfaces for thereby allowing the medial and lateral articular pads to pivot and rotate within the medial and lateral tray sockets. The first and second cushion members are preferably formed from a compressible, shock-absorbing, elastic material and are configured to absorb forces and impacts experienced by the medial and lateral articular pads.

Preferably, the medial articular pad includes a first pad cavity that is configured to receive the first cushion member and the first pivot bearing and the lateral articular pad comprises a second cavity that is configured to receive the second cushion member and the second pivot bearing.

Yet more preferably, the tibial component further includes a first bearing housing and a second bearing housing which can be received into the first and second pad cavities. The first bearing housing can include a first housing cavity that is configured to receive the first cushion member and the first pivot bearing and the second bearing housing can include a second housing cavity that is configured to receive the second cushion member and the second pivot bearing.

Preferably, the first pivot bearing includes a first hemi-spherical outer surface and a first bearing cavity and the second pivot bearing includes a second hemi-spherical outer surface and a second bearing cavity. The first and second hemi-spherical outer surfaces can be configured to engage the socket bottom surfaces and the first and second cushion members can be received into the first and second bearing cavities.

Preferably, the tibial component further comprises a first and a second bearing support pad. The first bearing support pad is supported on top of the first bottom surface and the first pivot bearing can be configured to engage and pivot, roll, and slide against the first bearing support pad. The second bearing support pad is supported on top of the second bottom surface and the second pivot bearing can be configured to engage and pivot, roll, and slide against the second bearing support pad. Yet more preferably, the first and second bearing support pads include first and second concave upper surfaces wherein the concave upper surfaces are slidingly engaged by the hemi-spherical outer surfaces of the pivot bearings.

Preferably, the tibial component further includes a first cushion base and a second cushion base. The first cushion base can be configured to line the bottom surface of the medial tray socket and the second cushion base can be configured to line the bottom surface of the lateral tray socket. In use, when the first and second cushion members are compressed by shocks or forces experienced by the medial and lateral articular pads, the medial and lateral articular pads engage the first and second cushion bases which can be configured to dampen and further absorb the shocks or forces to the articular pads.

Yet more preferably, the tibial component includes a first cushion sleeve and a second cushion sleeve. The first cushion sleeve can be configured to line the side and bottom surfaces of the medial tray socket and includes a first sleeve socket. The second cushion sleeve can be configured to line the side and bottom surfaces of the lateral tray socket and includes a second sleeve socket. In use, the medial articular pad is received into the first sleeve socket and the lateral articular pad is received into the second sleeve socket. The first and second cushion sleeves can be formed from a compressible, shock-absorbing elastic material and when the medial and lateral articular pads pivot or rotate within the first and second sleeve sockets, the medial and lateral articular pads engage and are cushioned by the first and second cushion sleeves.

In another embodiment, a tibial component of a prosthetic knee implant can comprise an implant tray having a medial tray socket and a lateral tray socket, a medial articular pad, a lateral articular pad, a medial cushion base, and a lateral cushion base. The medial articular pad is sized and shaped to fit into and is pivotably supported within the medial tray socket. The lateral articular pad is sized and shaped to fit into and is pivotably supported within the lateral tray socket. The medial cushion base lines the bottom surface of the medial tray socket and includes a first downwardly sloped portion formed at a posterior end thereof. The lateral cushion base lines the bottom surface of the lateral tray socket and includes a second downwardly sloped portion formed at a posterior end thereof.

In use, the medial cushion base is configured to absorb forces and impacts experienced by the medial articular pad and the lateral cushion base is configured to absorb forces and impacts experienced by the lateral articular pad. Additionally, when the medial and lateral articular pads pivot in a posterior direction, the posterior ends of the medial and lateral articular pads also pivot downwardly towards the first and second downwardly sloped portions.

Preferably, the implant tray also includes a depressed portion along the posterior edge of one or both of the medial and lateral tray sockets. Yet more preferably, the implant tray includes a depressed portion along the posterior edge of the lateral tray socket.

### BRIEF DESCRIPTION OF THE DRAWINGS

The above-mentioned and other features of this invention and the manner of attaining them will become more apparent, and the invention itself will be better understood by reference to the following description of the embodiments of the invention, taken in conjunction with the accompanying drawings, wherein:

FIG. 1 is a perspective view of a natural knee joint comprising the upper end of a tibia and the lower end of a femur;

FIG. 2 is a bottom perspective view of the femur shown in FIG. 1;

FIG. 3 is a top perspective view of the tibia shown in FIG. 1;

FIG. 4 is a perspective view of a prosthetic knee joint comprising the lower end of a femur with a femoral component secured thereto and the upper end of a tibia with a tibial component secured thereto;

FIG. 5 is a partially exploded perspective view of the tibia and tibial component shown in FIG. 4;

FIG. 6A is an exploded perspective view of the tibial component shown in FIGS. 4 and 5 having a bearing assembly with a viscoelastic polymer cushion;

FIG. 6B is an exploded perspective view of the tibial component shown in FIGS. 4 and 5 having bearing assembly with a coil spring;

FIG. 7 is a top plan view of the tibia and tibial component shown in FIGS. 4 and 5;

FIG. 8A is a cross-section view of the tibia and tibial component taken along the line A-A shown in FIG. 7, wherein the bearing assemblies include viscoelastic polymer cushion;

FIG. 8B is an exploded cross-section view of the tibial component shown in FIG. 8A;

FIG. 9A is a cross-section view of the tibia and tibial component taken along the line A-A shown in FIG. 7, wherein the bearing assemblies include coil springs;

FIG. 9B is an exploded cross-section view of the tibial component shown in FIG. 9A;

FIG. 10 is a front elevation view of the prosthetic knee joint shown in FIG. 4;

FIG. 11A is a cross-section view of the prosthetic knee joint taken along the line B-B shown in FIG. 10, wherein the prosthetic knee joint is fully extended;

FIG. 11B is a cross-section view of the prosthetic knee joint taken along the line B-B shown in FIG. 10, wherein the prosthetic knee joint is partially flexed;

FIG. 11C is a cross-section view of the prosthetic knee joint taken along the line B-B shown in FIG. 10, wherein the prosthetic knee joint is fully flexed;

FIG. 11D is a magnified detail view of Circled Detail 11D shown in FIG. 11C;

FIGS. 12A-C are cross-section views of the tibia and tibial component taken along the line C-C shown in FIG. 7 illustrating the tibial component's balancing of unevenly distributed forces between the medial and lateral sides of the tibial component; and,

FIGS. 13A-B are cross-section views of the tibia and tibial component taken along the line A-A shown in FIG. 7, wherein FIG. 13A illustrates the tibial component's absorption of small shocks/impact forces and FIG. 13B illustrates the tibial component's absorption of larger shocks/impact forces.

Corresponding reference characters indicate corresponding parts throughout several views. Although the exemplification set out herein illustrates certain embodiments of the invention, the embodiments disclosed below are not intended to be exhaustive or to be construed as limiting the scope of the invention to the precise form disclosed.

### DETAILED DESCRIPTION OF THE PREFERRED EMBODIMENTS

At the outset, the terms “medial”, “lateral”, “anterior”, and “posterior” are used herein to describe the relative positions and mobilities of various elements. Specifically, the term “medial” is used to refer to elements (or portions of an element) which are located closer to the midline of the human body or to refer to movements in a direction towards the midline of the human body. The term “lateral” is used to refer to elements (or portions of an element) which are located further away to the midline of the human body or to refer to movements in a direction away the midline of the human body. The term “anterior” is used to refer to elements (or portions of an element) which are located closer to the front of the human body or to refer to movements in a direction towards the front of the human body. The term “posterior” is used to refer to elements (or portions of an element) which are located closer to the back of the human body or to refer to movements in a direction towards the back of the human body.

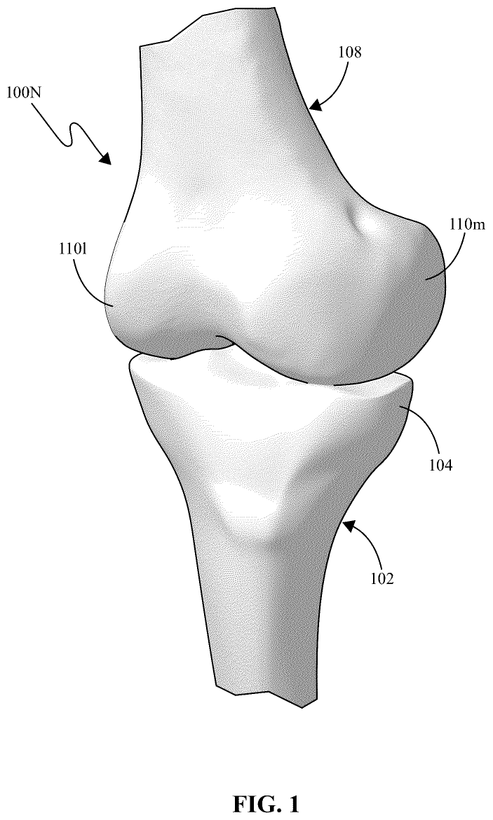

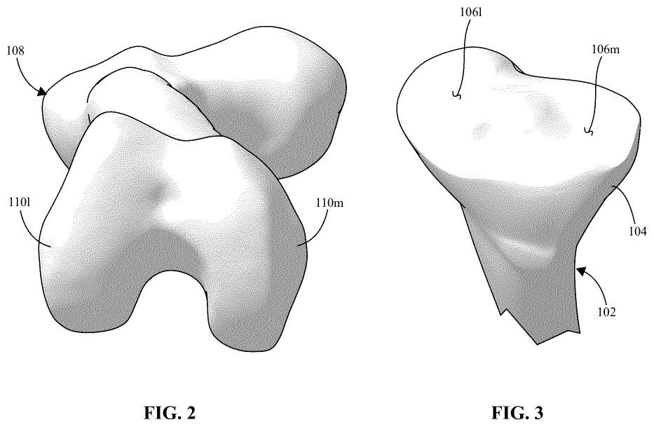

Referring initially to FIGS. 1-3, a natural knee joint 100N comprises the interface between the flat, shelf-like upper portion of a tibia 102, referred to as the tibial plateau 104, and the oblong, knuckle-like lower portions of a femur 108, referred to as the medial and lateral femoral condyles 110m, 110l. The tibial plateau 104 comprises a pair of concave articular surfaces which are referred to as the medial and lateral tibial condyles 106m, 106l. As the natural knee joint 100N flexes/bends and extends/straightens, the femoral condyles 110m, 110l engage and roll/pivot/slide on the tibial condyles 106m, 106l.

If the tibial plateau 104 and/or the femoral condyles 110m, 110l become damaged, for example, due to injury or disease, the mobility of the natural knee joint 100N can be impaired. If the damage is severe, the damaged tibial plateau 104 and femoral condyles 110m, 110l may need to be surgically removed or “resected” and replaced by prosthetic components which replicate the articular structures and functions of the knee joint.

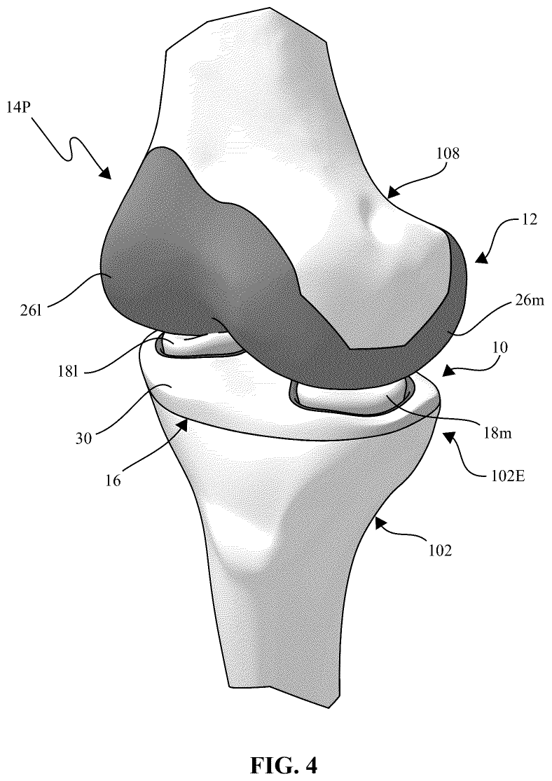

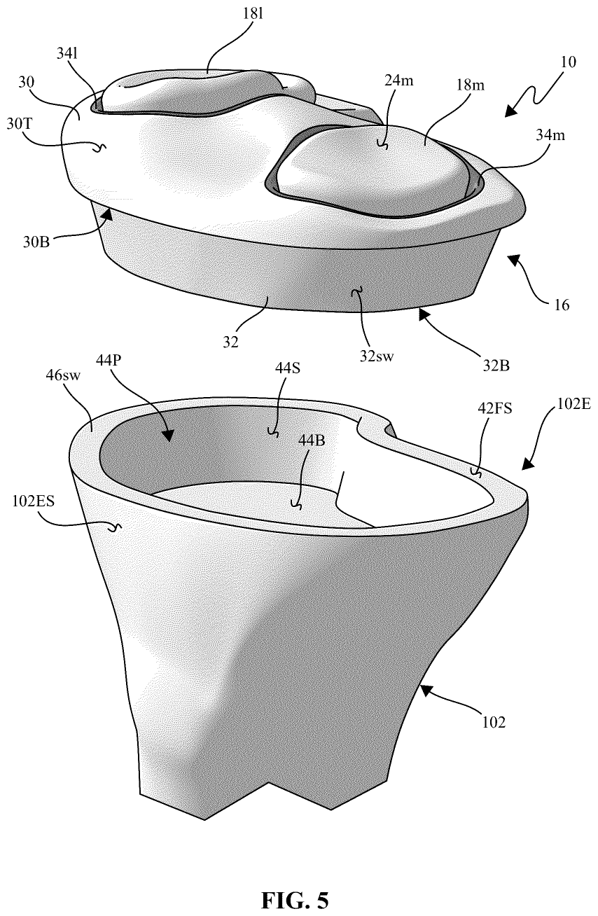

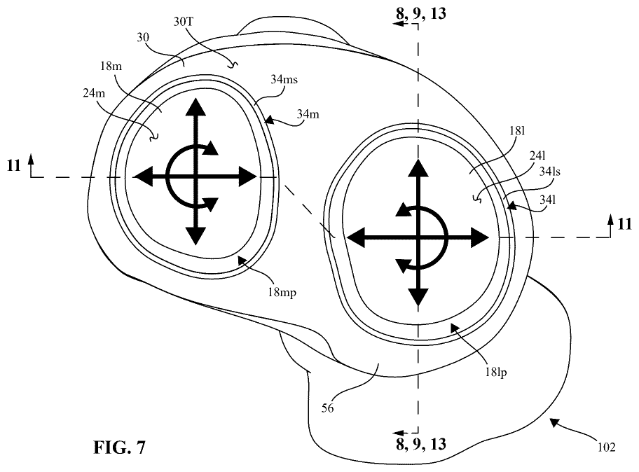

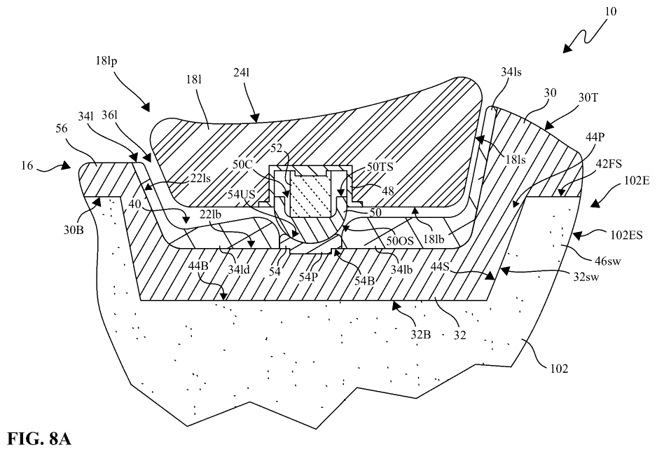

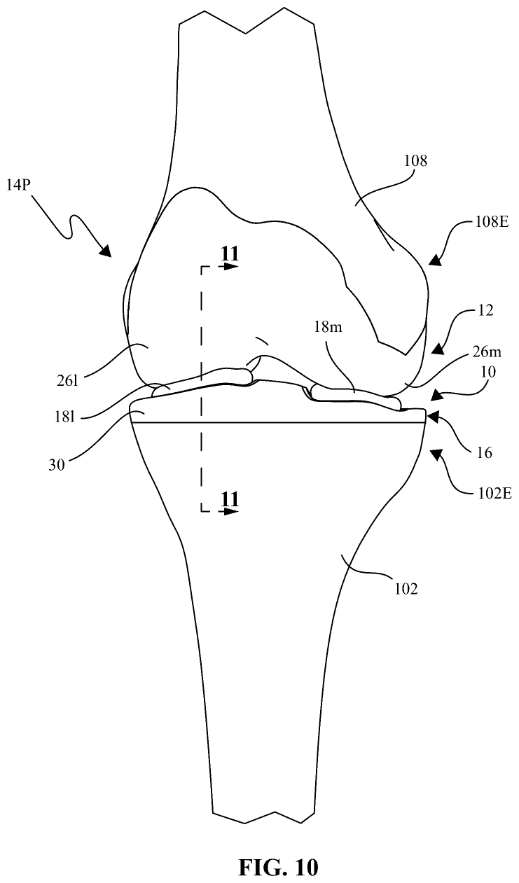

As shown in FIGS. 4-12, a prosthetic tibial component constructed in accordance with the principles of the present invention is shown and designated by the numeral 10. The tibial component 10 is adapted to be secured to the resected upper terminal end 102E of a tibia 102 and replicates the articular structures and functions of a tibial plateau 104. The tibial component 10 can be used together with a femoral component 12. The femoral component 12 is adapted to be secured to the resected lower terminal end 108E of a femur 108 and replicates the articular structures and functions of natural femoral condyles 110m, 110l. When used together, the tibial and femoral components 10, 12 form a prosthetic knee joint 14P which replicates the structures and functions of a natural knee joint 100N.

As will be described in greater detail below, the tibial component 10 comprises an implant tray 16, a medial articular pad 18m, a lateral articular pad 18l, and a pair of shock absorbing pivot assemblies 20. The implant tray 16 is adapted to be secured to the resected upper terminal end 102E of the tibia 102 and includes a pair of medial and lateral tray sockets 22m, 22l. The medial and lateral articular pads 18m, 18l are pivotably supported and suspended within the medial and lateral tray sockets 22m, 22l by the shock absorbing pivot assemblies 20 and comprise concave upper surfaces referred to as the medial and lateral artificial tibial condyles 24m, 24l. The medial and lateral artificial tibial condyles 24m, 24l are adapted to replicate the size and shape of the natural tibial condyles 106m, 106l and, preferably, are sized and shaped on a patient-by-patient basis to replicate each patient's unique anatomy. In use, the tibial component 10 acts like a natural tibial plateau 104 and supports the femoral component 12, and, hence, the femur 108, such that the forces experienced by the prosthetic knee joint 14P transfer downwardly through the tibial component 10 to the tibia 102.

The femoral component 12 can be a cap-like member that secures to and covers the resected lower terminal end 108E of a femur 108 and comprises a pair of oblong, knuckle-shaped protrusions referred to as the medial and lateral artificial femoral condyles 26m, 26l. The artificial femoral condyles 26m, 26l are configured to replicate the size and shape of the natural femoral condyles 110m, 110l. Preferably, the artificial femoral condyles 26m, 26l are sized and shaped on a patient-by-patient basis to replicate the patient's unique anatomy. As the prosthetic knee joint 14P flexes/bends and extends/straightens, the artificial femoral condyles 26m, 26l engage and roll/pivot/slide on the artificial tibial condyles 24m, 24l like the natural femoral condyles 110m, 110l engage and roll/pivot/slide on the natural tibial condyles 106m, 106l.

Preferably, the femoral component 12 is integrally formed from a high-strength, wear-resistant metallic material such as, for example, stainless steel, titanium, cobalt chrome, magnesium, and alloys comprising the foregoing, or a high-strength, wear-resistant ceramic material, such as, for example, alumina or zirconia based ceramic materials, to minimize wear/degradation of the artificial femoral condyles 26m, 26l during high impact loading and/or over a high number of load cycles.

As mentioned above, the tibial component 10 comprises an implant tray 16, a medial articular pad 18m, a lateral articular pad 18l, and a pair of shock absorbing pivot assemblies 20. The implant tray 16 is adapted to be secured to the resected upper terminal end 102E of the tibia 102 and comprises a tray cap 30, a tray base 32, and a pair of medial and lateral tray sockets 22m, 22l. The tray cap 30 is configured to replicate size and shape of the resected portion of the tibial plateau 104 and includes a planar cap bottom surface 30B and a cap top surface 30T. The tray base 32 extends downwardly from the cap bottom surface 30B and includes a base perimeter side wall 32sw and a base bottom surface 32B. Preferably, the implant tray 16 is integrally formed from a high-strength, wear-resistant metallic material such as, for example, stainless steel, titanium, cobalt chrome, magnesium, and alloys comprising the foregoing.

The medial and lateral tray sockets 22m, 22l are formed in the cap top surface 30T and extend downwardly through the tray cap 30 into the tray base 32. The tray sockets 22m, 22l comprise planar tray socket bottom surfaces 22mb, 22lb, tray socket side surfaces 22ms, 22ls, and tray socket openings 22mo, 22lo which open through the cap top surface 30T. The tray socket side surfaces 22ms, 22ls taper inwardly from the tray socket openings 22mo, 22lo, which are located at the widest part of the tray sockets 22m, 22l, to the planar tray socket bottom surfaces 22mb, 22lb, which are located at the narrowest part of the tray sockets 22m, 22l. The tray sockets 22m, 22l are sized to ensure that the implant tray 16 has sufficient structural strength to support the forces transferred thereto through the shock absorbing pivot assemblies 20.

The medial and lateral articular pads 18m, 18l are received into the tray sockets 22m, 22l and are pivotably supported and suspended therein by the shock absorbing pivot assemblies 20. The articular pads 18m, 18l comprise planar pad bottom surfaces 18mb, 18lb, vertical pad side surfaces 18ms, 18ls, and concave upper pad surfaces, referred to as the medial and lateral artificial tibial condyles 24m, 24l. The artificial tibial condyles 24m, 24l are sized and shaped to replicate the natural tibial condyles 106m, 106l and are adapted to slidingly engage the artificial femoral condyles 26m, 26l. The vertical pad side surfaces 18ms, 18ls are, preferably, tapered to match the shape of the tray socket side surfaces 22ms, 22ls.

Preferably, the articular pads 18m, 18l are integrally formed from a high-strength, high wear-resistance polymer material such as, for example, polytetrafluoroethylene (PTFE), ultra-high molecular weight polyethylene (UHMWPE), or polyetheretherketone (PEEK), or a high-strength, wear-resistant ceramic material such as, for example, alumina or zirconia based ceramic materials to minimize wear/degradation of the articular pads 18m, 18l during high impact loading and/or over a high number of load cycles.

Preferably, a pair of cushion sleeves 34m, 34l line the interior surfaces of the tray sockets 22m, 22l. The cushion sleeves 34m, 34l are adapted to support and absorb shocks and impacts to the articular pads 18m, 18l like the menisci (not shown) of a natural knee joint 100N. Preferably, the cushion sleeves 34m, 34l are constructed from a shock-absorbing, elastic material, and yet more preferably, from a shock-absorbing, viscoelastic material, such as, for example, viscoelastic carbon nanotubes, polyethylene fiber reinforced polycarbonate-urethane, rubber, silicone rubber, and other shock-absorbing, viscoelastic materials.

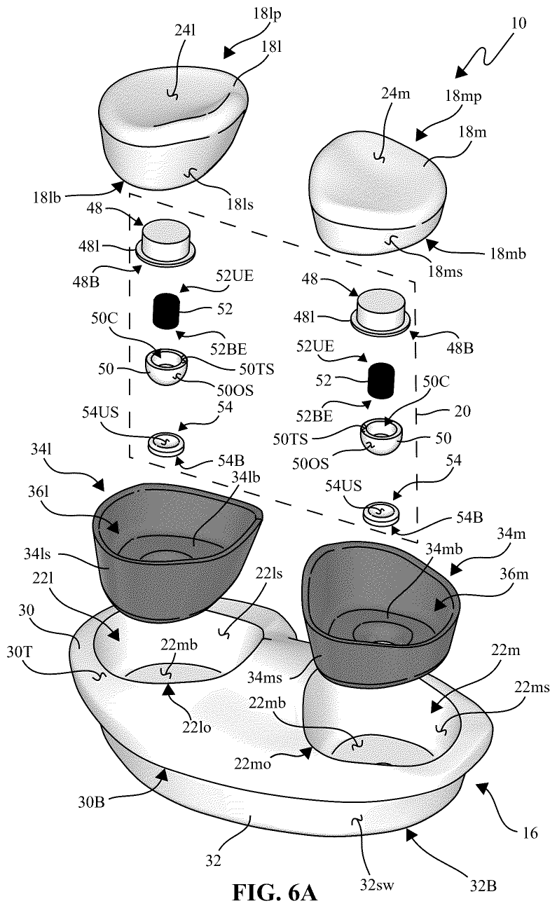

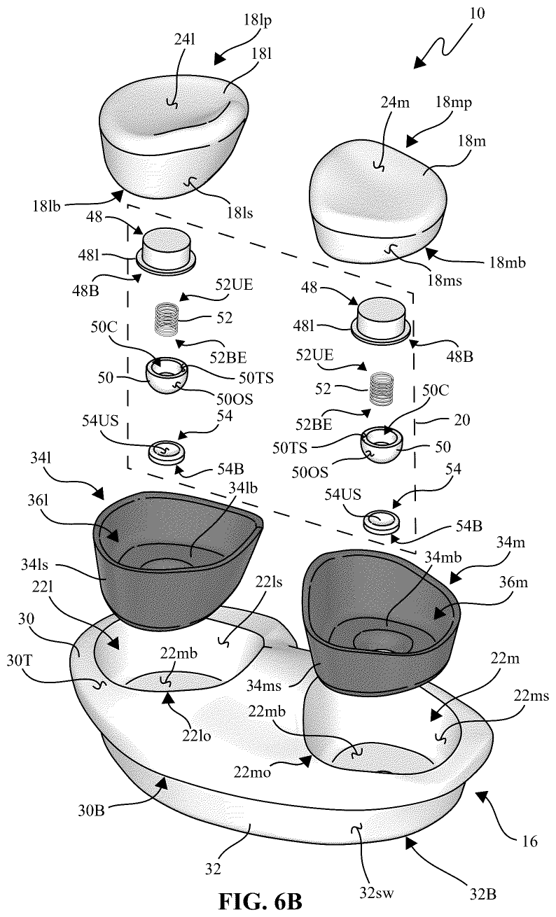

As best seen in FIGS. 6A-B, 8B, and 9B, the cushion sleeves 34m, 34l comprise cushion sleeve bases 34mb, 34lb, vertical cushion sleeve side walls 34ms, 34ls, and cushion sleeve sockets 36m, 36l. The cushion sleeve bases 34mb, 34lb line the socket bottom surfaces 22mb, 22lb. The cushion sleeve side walls 34ms, 34ls extend upwardly from the cushion sleeve bases 34mb, 34lb and line the socket side surfaces 22ms, 22ls. The cushion sleeve sockets 36m, 36l are defined between the cushion sleeve bases 34mb, 34lb and the cylindrical cushion sleeve side walls 34ms, 34ls. The cushion sleeve sockets 36m, 36l are adapted to receive the articular pads 18m, 18l and are sized such that the articular pads 18m, 18l can pivot and/or rotate therein.

As mentioned above, the implant tray 16 is adapted to be secured to the resected upper terminal end 102E of the tibia 102. Preferably, the implant tray 16 is adapted to be secured within a fixation pocket 44P formed at the resected upper terminal end 102E of the tibia 102. Specifically, when the damaged tibial plateau 104 is surgically removed/resected, a planar tibial fixation surface 42FS is formed at the upper terminal end 102E of the tibia 102. A tibial fixation pocket 44P is formed in the tibial fixation surface 42FS extending downwardly into the bone tissue of the tibia 102 and a tibial perimeter support wall 46sw is defined between the tibial fixation pocket 44P, the planar tibial fixation surface 42FS, and an exterior perimeter surface 102ES of the tibia 102. The tibial fixation pocket 44P comprises a fixation pocket perimeter side surface 44S and a fixation pocket bottom surface 44B. The tray base 32 is, preferably, adapted to be press-fit into the tibial fixation pocket 44P with the base perimeter side wall 32sw abutting the fixation pocket perimeter side surface 44S and the base bottom surface 32B abutting the fixation pocket bottom surface 44B. The tray cap 30 is supported above the tibial perimeter support wall 46sw with the cap bottom surface 30B abutting the planar tibial fixation surface 42FS.

Preferably, the cap bottom surface 30B, the base perimeter side wall 32sw, and the base bottom surface 32B are porous and are coated with a bioactive coating material, such as, for example: bioceramics including, for example, metal oxides, calcium phosphates, silicates, glasses, glass-ceramics, and carbon nanotube reinforced ceramics; extracellular matrix proteins; biological peptides; hydroxyapatite; and other bioactive coating materials. The bioactive coating material is adapted to promote the growth of bone tissue into pores of the cap bottom surface 30B, the base perimeter side wall 32sw, and the base bottom surface 32B such that the implant tray 16 fuses to the tibia 102. The bioactive coating material can be used together with bone cement, as necessary or desirable, to secure the implant tray 16 to the tibia 102.

Preferably, the tibial fixation pocket 44P is formed in the “spongy” cancellous bone of the tibia 102 and the tibial perimeter side wall 46sw is formed by the hard, cortical bone of the tibia 102. In this regard, the ends of long bones, such as, for example, tibias 102 and femurs 108, comprise two, different layers of bone tissue: an outer layer of harder, compact bone (referred to as cortical bone, compact tissue, hard bone, or compact bone) and an inner layer of softer, porous bone tissue (referred to as cancellous bone, cancellous tissue, spongy bone, or trabecular bone). The harder, cortical bone tissue provides strength to the tibia 102 and protects the softer, cancellous bone. Preferably, the surgical removal/resection of the tibial plateau 104 is performed whereby as much of the cortical bone tissue is preserved as possible to maintain the strength and structural integrity of the tibia 102.

The softer, cancellous bone has greater blood flow than the harder, cortical bone, and, therefore, tends to grow/regenerate faster. By forming the fixation pocket 44P within the cancellous bone of the tibia 102, bone tissue grows more quickly into pores of the base perimeter side wall 32sw and the base bottom surface 32B for thereby fusing the implant tray 16 to the tibia 102. Additionally, the tibial perimeter side wall 46sw, which is formed from the hard, cortical bone of the tibia 102, acts as a stable, rigid support for the implant tray 16 providing rotational and vertical stability and prevents the implant tray from moving vertically within the fixation pocket 44P.

As best seen in FIGS. 6, 8, and 9, the shock absorbing pivot assemblies 20, which support the articular pads 18m, 18l within the tray sockets 22m, 22l, comprise a cylindrical bearing housing 48, a pivot bearing 50, a cushion member 52, and a support pad 54. The bearing housings 48 are secured to the articular pads 18m, 18l and comprise a central, cylindrical housing cavity 48C which opens through a bottom end 48B of the housing 48. The housing cavities 48C comprise a planar base surface 48CB and a cylindrical side surface 48CS.

Preferably, the bearing housings 48 are secured to the articular pads 18m, 18l by press-fitting, adhering, or otherwise mounting or securing the bearing housings 48 into cylindrical pad cavities 18C formed in the planar pad bottom surfaces 18mb, 18lb. Yet more preferably, the bearing housings 48 further include an annular ledge 48L which extends outwardly from the housing 48 adjacent to the housing bottom end 48B. The annular ledges 48L engage and embed into the planar pad bottom surfaces 18mb, 18lb and are adapted to distribute the forces transferred between the housings 48 and the articular pads 18m, 18l.

The pivot bearings 50 comprise a semi-spherical bearing outer surface 50OS, a planar bearing top surface 50TS, and a bearing cavity 50C which extends downwardly into the planar bearing top surface 50TS. The pivot bearings 50 are sized to be received into the cylindrical housing cavities 48C with the semi-spherical bearing outer surfaces 50OS facing downwardly towards the bearing support pads 54. In use, the semi-spherical bearing outer surfaces 50OS slidingly engage the concave upper surfaces 54US of the bearing support pads 54.

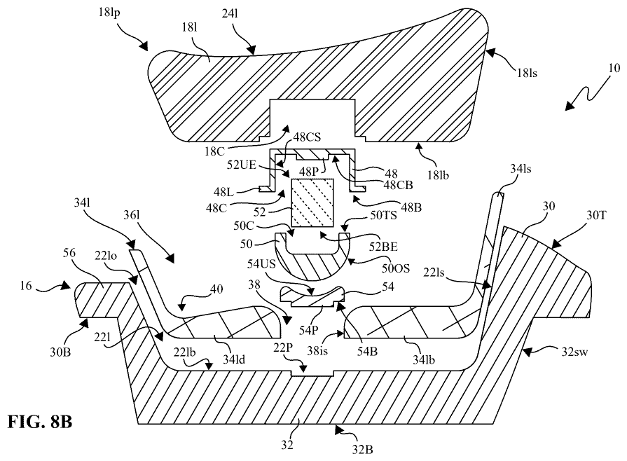

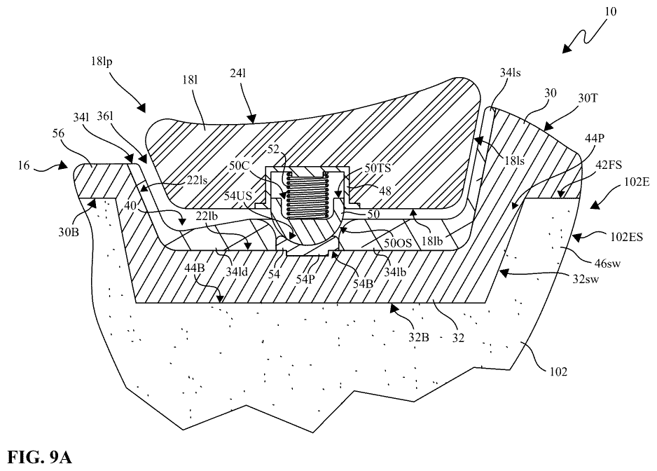

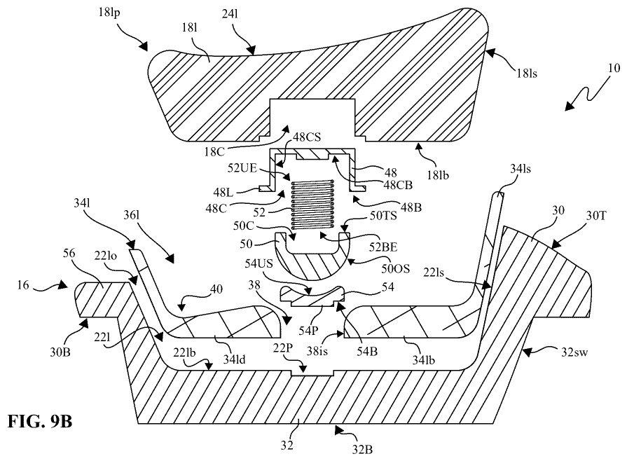

The cushion members 52 are sandwiched between the bearing housings 48 and the pivot bearings 50 and comprise an upper end 52UE which engages the planar housing cavity base surface 48CB and a bottom end 52BE which is received into the bearing cavity 50C. The cushion members 52 are adapted to support and suspend the articular pads 18m, 18l above the tray socket bottom surfaces 22mb, 22lb and, like the cushion sleeves, absorb shocks and impact forces experienced by the knee joint. Preferably, the cushion members 52 can be helical springs (FIGS. 6B and 9A-B), viscoelastic polymer cushions (FIGS. 6A, 8A-B, 11A-D, 12A-C, and 13A-B), belleville washers (not shown), disc springs (not shown), or other types of shock-absorbing springs or cushions.

Preferably, the bearing housings 48 include a cushion locating protrusion 48P. The cushion locating protrusions 48P extend downwardly from the housing cavity base surfaces 48CB and are adapted to engage the cushion upper ends 52UE. Together, the bearing cavities 50C and the cushion locating protrusions 48P engage and align the cushion members 52 within the housing cavities 48C.

The bearing support pads 54 are adapted to support the pivot bearings 50 and distribute forces across the planar tray socket bottom surfaces 22mb, 22lb. The bearing support pads 54 comprise concave bearing support upper surfaces 54US and planar bearing support bottom surfaces 54B. As mentioned above, the concave bearing support upper surfaces 54US are adapted to slidingly engage the semi-spherical bearing outer surfaces 50OS. The planar bearing support pad bottom surfaces 54B are adapted to abut and distribute forces across the planar socket bottom surfaces 22mb, 22lb.

Preferably, the cushion sleeves 34m, 34l include cushion sleeve bores 38 which extend vertically through the cushion sleeve bases 34mb, 34lb. The cushion sleeve bores 38 are adapted to receive the bearing support pads 54 therethrough and are sized such that bearing support pads 54 engage and are prevented from sliding across the planar socket bottom surfaces 22mb, 22lb by the interior surfaces 38is of the cushion sleeve bores 38.

Preferably, the bearing support pads 54 further include pad locating protrusions 54P which extend downwardly from the planar bearing support bottom surfaces 54B. The tray sockets 22m, 22l include corresponding locating pockets 22P which formed in the planar socket bottom surfaces 22mb, 22lb and are adapted to receive the pad locating protrusions 54P for thereby preventing the bearing support pads 54 from sliding across the planar socket bottom surfaces 22mb, 22lb.

In use, the tibial and femoral components 10, 12 are adapted to replicate the functions of the natural tibial plateau 104 and the natural femoral condyles 110m, 110l, respectively. Specifically, in a natural knee joint 100N, the natural femoral condyles 110m, 110l engage the natural tibial condyles 106m, 106l and are supported above the tibial plateau 104. As the natural knee joint 100N flexes/bends and extends/straightens, the natural femoral condyles 110m, 110l roll/pivot in the anterior/posterior directions and slide against the natural tibial condyles 106m, 106l.

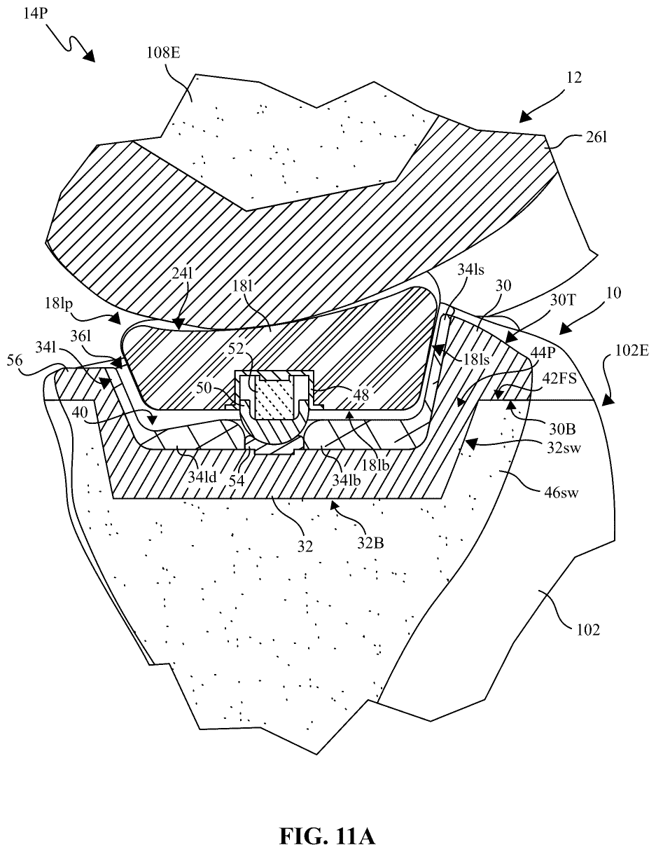

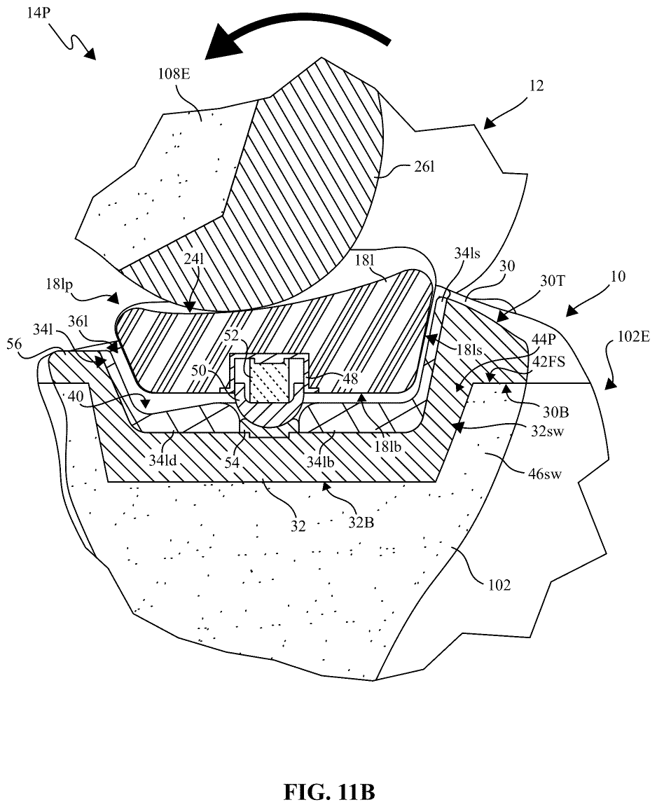

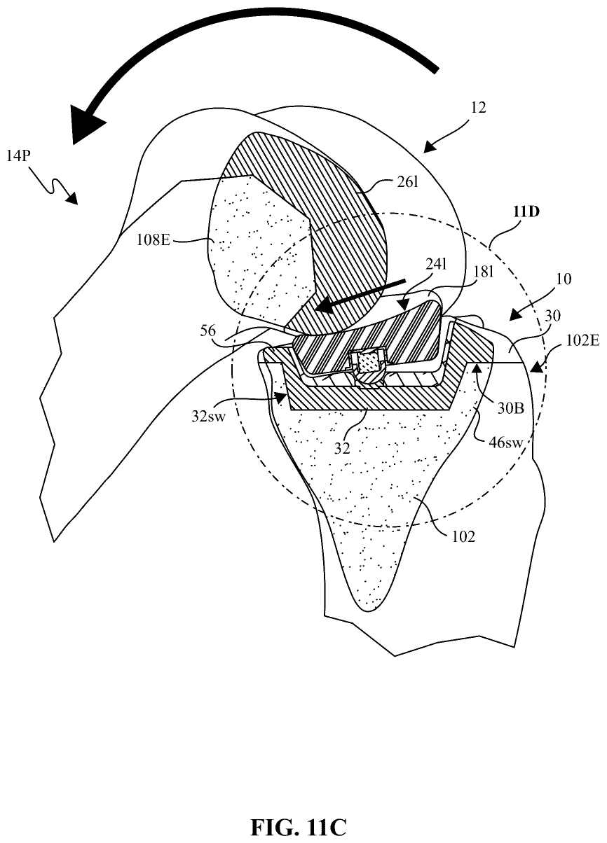

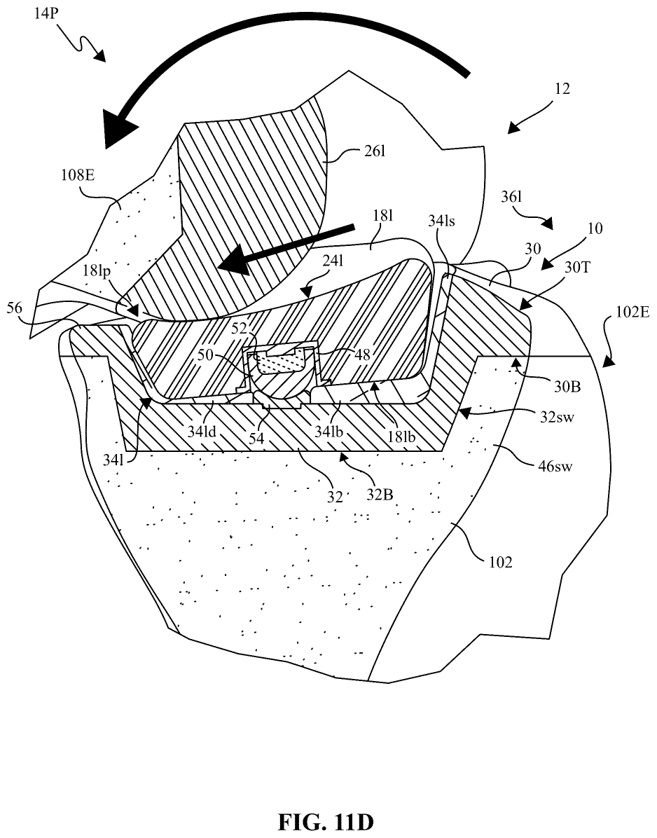

In the present invention, when the prosthetic knee joint 14P is fully extended/straightened (FIGS. 4, 10, and 11A), the artificial femoral condyles 26m, 26l engage the artificial tibial condyles 24m, 24l and are supported above the articular pads 18m, 18l. As the prosthetic knee joint 14P flexes/bends, the artificial femoral condyles 26m, 26l roll/pivot in the posterior direction and slide against the artificial tibial condyles 24m, 24l. As the prosthetic knee joint 14P extends/straightens from a flexed/bent position (FIGS. 11B-D), the artificial femoral condyles 26m, 26l roll/pivot in the anterior direction and slide against the artificial tibial condyles 24m, 24l.

When a natural knee joint 100N approaches the limit of its range of flexion (i.e., when the calf (not shown) approaches the back of the thigh (not shown)), the shape of the natural condyles 106m, 106l, 110m, 110l and the geometry of the human leg cause the femur 108 to pivot in the medial direction, which, in turn, causes the lateral femoral condyle 110l to slide/translate towards the posterior end of the lateral tibial condyle 106l. This pivoting of the femur 108 and the posterior translation of the lateral femoral condyle 110l allows the natural knee joint 100N to maintain proper alignment with the hip joint (not shown) as the knee joint approaches the end of its range of motion.

As best seen in FIGS. 11A-D, the tibial and femoral components 10, 12 are adapted to replicate this pivoting of the femur 108. Specifically, by configuring the artificial condyles 24m, 24l, 26m, 26l to exactly replicate the size and shape of the natural condyles 106m, 106l, 110m, 110l, as the prosthetic knee joint 14P flexes/bends, the size and shape artificial condyles and the geometry of the human leg cause the femur 108 to pivot such that the lateral artificial femoral condyle 26l slides/translates towards the posterior end of the lateral artificial tibial condyle 24l. Like a natural knee joint 100N, the posterior translation of the lateral artificial femoral condyle 26l allows the prosthetic knee joint 14P to maintain proper alignment with the hip joint.

It should be noted that, in both a natural knee joint 100N and the prosthetic knee joint 14P, as the femur 108 pivots in the medial direction, the medial femoral condyles 110m, 26m also slide/translate towards the posterior ends of the medial tibial condyles 106m, 24m. However, the medial femoral condyles 110m, 26m typically translate a much shorter distance than the lateral femoral condyles 110l, 26l.

In addition to replicating the functions of the tibial plateau 104, the tibial component 10 is also adapted to: (i) maintain contact and proper alignment between the artificial tibial and femoral condyles 24m, 24l, 26m, 26l, (ii) balance the forces experienced by the prosthetic knee joint 14P between the lateral and medial pairs of condyles, and (iii) absorb shocks/impact forces experienced by the prosthetic knee joint 14P. Specifically, as mentioned hereinabove, the shock absorbing pivot assemblies 20 pivotably support and suspend the articular pads 18m, 18l within the tray sockets 22m, 22l. The shock absorbing pivot assemblies 20, the articular pads 18m, 18l, the tray sockets 22m, 22l, and the cushion sleeves 34m, 34l are adapted such that the articular pads 18m, 18l can: (1) pivot in the anterior/posterior directions; (2) pivot in the medial/lateral directions; (3) rotate in the clockwise and counter clockwise directions); and (4) retract downwardly into or extend upwardly out of the tray sockets 22m, 22l.

With respect to maintaining contact and proper alignment between the artificial tibial and femoral condyles 24m, 24l, 26m, 26l, as shown in FIGS. 11C-D, when the prosthetic knee joint 14P approaches the end of its range of flexion (i.e., when the calf (not shown) approaches the back of the thigh (not shown)), the artificial femoral condyles 26m, 26l slide towards the posterior ends 18mp, 48lp of the articular pads 18m, 18l. This posterior translation causes the articular pads 18m, 18l to pivot in the posterior direction. By allowing the articular pads 18m, 18l to pivot in the posterior direction, the artificial femoral condyles 26m, 26l remain in contact with the artificial tibial condyles 24m, 24l and are prevented from sliding, rolling, or falling over the pad posterior ends 18mp, 18lp as the prosthetic knee joint 14P approaches the end of its range of flexion.

Additionally, as the articular pads 18m, 18l pivot in the posterior direction, the articular pad posterior ends 18mp, 18lp pivot downwardly towards the tray cap 30 and the posterior ends of the articular pad bottom surfaces 18mb, 18lb pivot downwardly towards the cushion sleeve bases 34mb, 34lb. This downward pivoting of the articular pad posterior ends 18mp, 18lp allows the artificial femoral condyles 26m, 26l, which have translated towards the articular pad posterior ends 18mp, 18lp, to shift/translate downwardly towards the tray cap 30 which reduces the tension placed on the patellar tendon as the prosthetic knee joint 14P bends and thereby allows the prosthetic knee joint 14P to achieve deeper flexion.

In this regard, traditional tibial components do not include articular pads which pivot downwardly as the knee joint flexes/bends. Thus, as the traditional prosthetic knee joint bends, the anterior side of the femur 108, whereat the femoral end of the patellar tendon (not show) is connected, shifts upwardly away from the tibia 102 and places the patellar tendon under tension. This tension increases as the traditional prosthetic knee joint flexes/bends and, eventually, prevents the traditional prosthetic knee joint from continuing to flex/bend. Therefore, by using tibial components 10 constructed in accordance with the present invention, the prosthetic knee joint 14P can be adapted to achieve a greater range of motion than a traditional prosthetic knee joint and, potentially, a greater range of motion than a natural knee joint 100N.

Preferably, as best seen in FIGS. 8, 9, and 11, the posterior ends of the cushion sleeve bases 34mb, 34lb include downwardly sloped portions 34md, 34ld whereby a wedge-shaped pocket 40 is formed at the posterior ends of the sleeve sockets 36m, 36l. When the articular pads 18m, 18l pivot in the posterior direction and the articular pad posterior ends 18mp, 18lp pivot downwardly towards the tray cap 30, the articular pad bottom surfaces 18mb, 18lb pivot downwardly and engage and compress the cushion sleeve bases 34mb, 34lb. By configuring the cushion sleeve bases 34mb, 34lb such that pockets 40 are formed at the posterior ends of the sleeve sockets 36m, 36l, the articular pads 18m, 18l able to pivot further downwardly before they engaging the wedge-shaped depressed portions 34md, 34ld. This, in turn, allows the prosthetic knee joint 14P to achieve yet deeper flexion.

Preferably, the tray cap 30 includes a depressed portion 56 along the posterior edge of one or both of the tray sockets 22m, 22l. For example, as best seen in FIG. 11D, a depressed portion 56 is formed along the posterior edge of the lateral tray socket 22l. The depressed portions 56 are adapted such that, as the posterior ends 18mp, 18lp of the articular pads 18m, 18l pivot downwardly towards the tray cap 30 and the lateral femoral condyle 26l shifts/translates towards the posterior end of the lateral tibial condyle 24l, the femoral condyles 26mm 26l do not engage or rub against the tray cap 30.

Of course, as the articular pads 18m, 18l pivot in the posterior direction, the posterior ends 18mp, 18lp thereof eventually engage the cushion sleeve side walls 34ms, 34ls. In this regard, the cushion sleeve side walls 34ms, 34ls prevent the articular pads 18m, 18l from striking the tray socket side surfaces 22ms, 22ls and act as cushions which compress and absorb the impact of the articular pads 18m, 18l thereagainst.

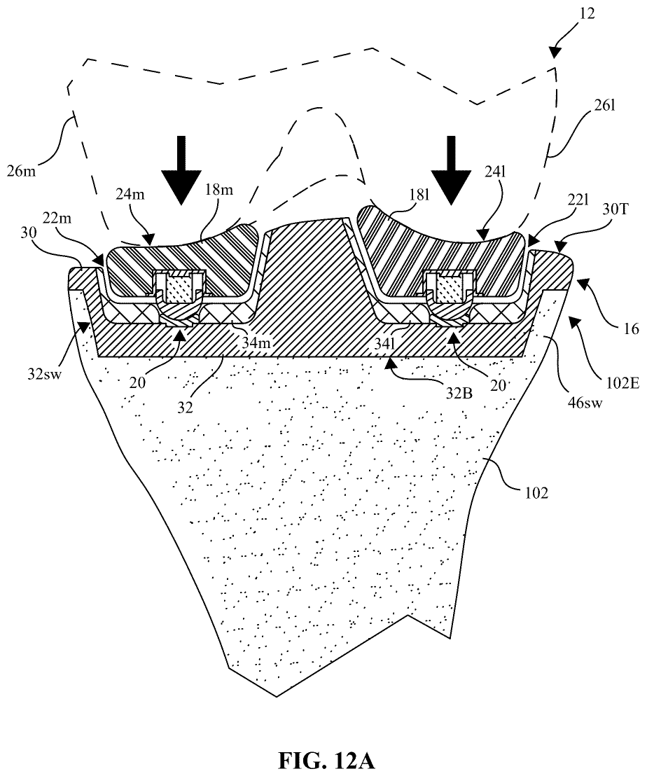

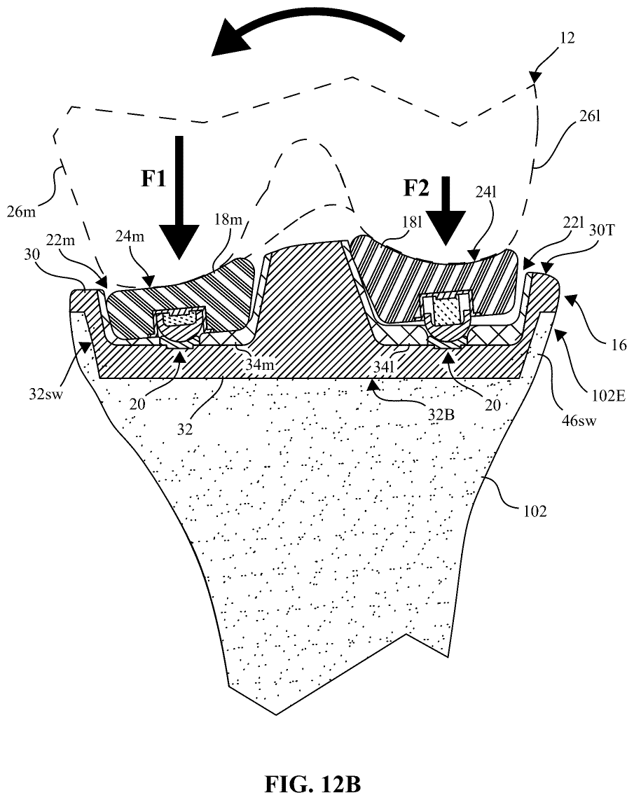

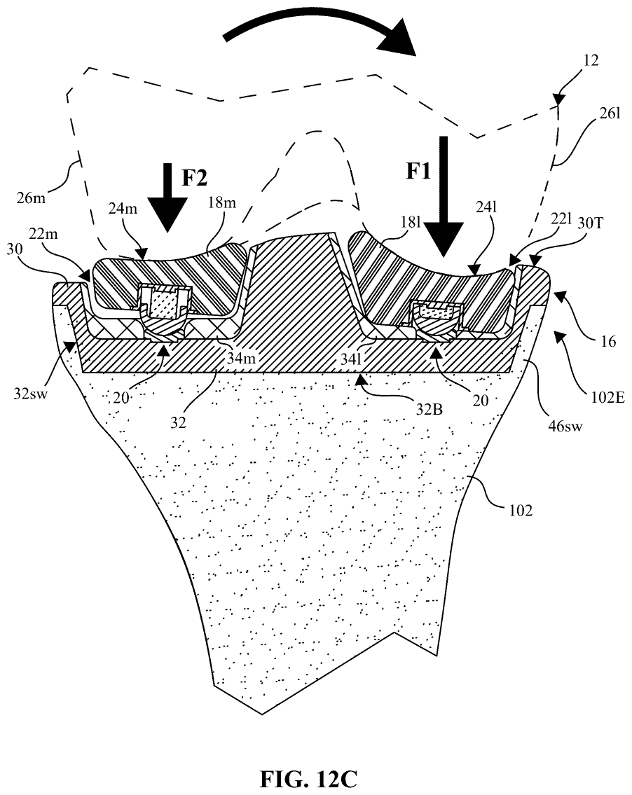

With respect to balancing the forces experienced by the prosthetic knee joint 14P, as shown in FIGS. 12A-C, if the forces experienced by the prosthetic knee joint 14P are distributed unevenly between medial and lateral sides of the tibial component 10, the articular pads 18m, 18l will pivot or tilt in the direction of the larger force to compensate for the uneven distribution. For example, if a larger force F1 is experienced by the medial articular pad 18m, the articular pads 18m, 18l will pivot or tilt in the medial direction and the medial articular pad 18l will retract downwardly into the medial tray socket 22l. (See FIG. 12B). Conversely, if a larger force F1 is experienced by the lateral articular pad 18l and a smaller force F2 is experienced by the medial articular pad 18m, the articular pads 18m, 18l will pivot or tilt in the lateral direction and the lateral articular pad 18l will retract downwardly into the lateral tray socket 22l. (See FIG. 12C).

As the articular pads 18m, 18l pivot/tilt and retract downwardly into the tray sockets 22m, 22l, the forces F1 and F2 redistribute more evenly between the articular pads 18m, 18l. This redistribution prevents excessive, uneven loading between the medial and lateral sides of the prosthetic knee joint 14P, which, in turn, reduces wear and tear on artificial femoral condyles 26m, 26l and the articular pads 18m, 18l.

With respect to absorbing shocks and impact forces, in a natural knee joint 100N, shocks/impact forces are absorbed by a pair of C-shaped cartilage structures called menisci (not shown) which are sandwiched between the tibial and femoral condyles. In the present invention, the cushion members 52 and the cushion sleeves 34m, 34l are adapted to replicate the shock absorbing function of the natural menisci. Specifically, when the prosthetic knee joint 14P experiences a shock/impact force, the articular pads 18m, 18l retract into the tray sockets 22m, 22l and the cushion members 52 and cushion sleeve bases 34mb, 34lb compress to absorb the shock/impact forces.

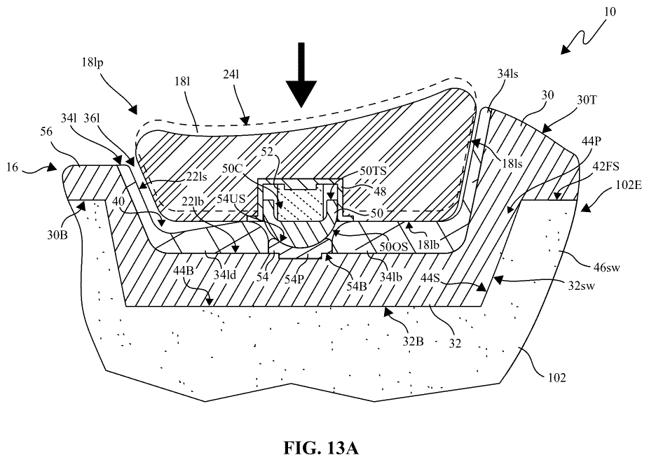

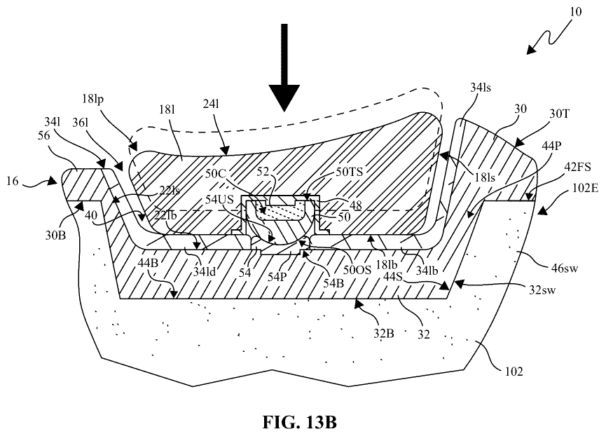

Preferably, the cushion members 52 and the cushion sleeve bases 34mb, 34lb act as a two-stage cushion/damper system for absorbing shocks/impact forces experienced by the prosthetic knee joint 14P. Specifically, when the prosthetic knee joint 14P experiences small shocks/impacts, for example, during ordinary walking, the cushion members 52 are adapted to compress and absorb energy from the shocks/impacts while supporting the articular pads 18m, 18l above the cushion sleeve bases 34mb, 34lb. (See FIG. 13A). When the knee joint 14P experiences larger shocks/impacts, for example, during jogging or jumping activities, both the cushion members 52 and the cushion sleeves 34m, 34l compress and absorb shocks/impact forces. (See FIG. 13B).

Preferably, except as otherwise set forth herein, the components of the tibial component 10 are formed from a high-strength, high wear resistant metallic material such as, for example, steel, stainless steel, titanium, cobalt chrome, magnesium, and alloys comprising the foregoing, or a high-strength, wear-resistant ceramic material such as, for example, alumina or zirconia based ceramic materials. Preferably, except as otherwise set forth herein, the components of the tibial component 10 are formed by casting, molding, machining, or otherwise shaping or forming from a unitary material.

While this invention has been described as having an exemplary design, the present invention may be further modified within the spirit and scope of this disclosure. This application is therefore intended to cover any variations, uses, or adaptations of the invention using its general principles.

What is claimed is:

1. A tibial component of a prosthetic knee implant, the tibial component comprising:
an implant tray having a medial tray socket and a lateral tray socket, the medial tray socket having a first socket bottom surface and the lateral tray socket having a second socket bottom surface;
a medial articular pad that is sized and shaped to fit into the medial tray socket;
a lateral articular pad that is sized and shaped to fit into the lateral tray socket;
a first and a second pivot bearing; and
a first and a second cushion member;
wherein the medial articular pad is supported within the medial tray socket by the first pivot bearing which engages the first socket bottom surface and the first cushion member which is sandwiched between the first pivot bearing and the medial articular pad;
wherein the lateral articular pad is supported within the lateral tray socket by the second pivot bearing which engages the second socket bottom surface and the second cushion member which is sandwiched between the second pivot bearing and the lateral articular pad;
wherein the first and second pivot bearings pivot, roll, and slide against the first and second socket bottom surfaces and thereby allow the medial and lateral articular pads to pivot and rotate within the medial and lateral tray sockets; and,
wherein the first and second cushion members absorb forces and impacts experienced by the medial and lateral articular pads.

2. The tibial component of claim 1, wherein the first and second cushion members are formed from a compressible, shock-absorbing, elastic material.

3. The tibial component of claim 1, wherein the first and second cushion members are springs.

4. The tibial component of claim 1, wherein the medial articular pad comprises a first pad cavity and the lateral articular pad comprises a second cavity, and wherein the first cushion member and the first pivot bearing are received into the first cavity and the second cushion member and the second pivot bearing are received into the second cavity.

5. The tibial component of claim 1 further comprising a first bearing housing and a second bearing housing, the first bearing housing having a first housing cavity and the second bearing housing having a second housing cavity, wherein the medial articular pad comprises a first pad cavity and the lateral articular pad comprises a second cavity, wherein the first bearing housing is received into the first pad cavity and the second bearing housing is received into the second pad cavity, and wherein the first cushion member and the first pivot bearing are received into the first housing cavity and the second cushion member and the second pivot bearing are received into the second housing cavity.

6. The tibial component of claim 1, wherein the first pivot bearing includes a first hemi-spherical outer surface and a first bearing cavity and the second pivot bearing includes a second hemi-spherical outer surface and a second bearing cavity, wherein the first and second hemi-spherical outer surfaces engage the first and second bottom surfaces, and wherein the first and second cushion members are received into the first and second bearing cavities.

7. The tibial component of claim 1 further comprising a first and a second bearing support pad, wherein the first bearing support pad is supported on top of the first bottom surface and the first pivot bearing engages and pivots, rolls, and slides against the first bearing support pad, and wherein the second bearing support pad is supported on top of the second bottom surface and the second pivot bearing engages and pivots, rolls, and slides against the second bearing support pad.

8. The tibial component of claim 7, wherein the first pivot bearing includes a first hemi-spherical outer surface and the second pivot bearing includes a second hemi-spherical outer surface, wherein the first bearing support pad includes a first concave upper surface and the second bearing support pad includes a second concave upper surface, and wherein the first hemi-spherical outer surface slidingly engages the first concave upper surface and the second hemi-spherical outer surface slidingly engages the second concave upper surface.

9. The tibial component of claim 8 further comprising a first bearing housing and a second bearing housing, the first bearing housing having a first housing cavity and the second bearing housing having a second housing cavity, wherein the medial articular pad comprises a first pad cavity and the lateral articular pad comprises a second cavity, wherein the first bearing housing is received into the first pad cavity and the second bearing housing is received into the second pad cavity, and wherein the first cushion member and the first pivot bearing are received into the first housing cavity and the second cushion member and the second pivot bearing are received into the second housing cavity.

10. The tibial component of claim 9, wherein the first pivot bearing includes a first bearing cavity and the second pivot bearing includes a second bearing cavity, and wherein the first and second cushion members are received into the first and second bearing cavities.

11. The tibial component of claim 1 further comprising a first cushion base and a second cushion base, wherein the first cushion base lines the first bottom surface and the second cushion base lines the second bottom surface, and wherein when the first and second cushion members are compressed by shocks or forces experienced by the medial and lateral articular pads, the medial and lateral articular pads engage the first and second cushion bases which dampen and further absorb the shocks or forces.

12. The tibial component of claim 1 further comprising a first cushion sleeve and a second cushion sleeve, wherein the medial tray socket includes a first side surface and the lateral tray socket includes a second side surface, wherein the first cushion sleeve lines the first side surface and the first bottom surface and includes a first sleeve socket, wherein the second cushion sleeve lines the second side surface and the second bottom surface and includes a second sleeve socket, and wherein the medial articular pad is received into the first sleeve socket and the lateral articular pad is received into the second sleeve socket.

13. The tibial component of claim 12, wherein the first and second cushion sleeves are formed from a compressible, shock-absorbing elastic material, and wherein when the medial and lateral articular pads pivot or rotate within the first and second sleeve sockets, the medial and lateral articular pads engage and are cushioned by the first and second cushion sleeves.

14. A tibial component of a prosthetic knee implant, the tibial component comprising:
an implant tray having a medial tray socket and a lateral tray socket, the medial tray socket having a first bottom surface and the lateral tray socket having a second bottom surface;
a medial articular pad that is sized and shaped to fit into and is pivotably supported within the medial tray socket, the medial articular pad having a first posterior end;
a lateral articular pad that is sized and shaped to fit into and is pivotably supported within the lateral tray socket, the lateral articular pad having a second posterior end;
a medial cushion base that lines the first bottom surface, the medial cushion base having a first downwardly sloped portion formed at a posterior end of the medial cushion base; and
a lateral cushion base that lines the second bottom surface, the lateral cushion base having a second downwardly sloped portion formed at a posterior end of the lateral cushion base;
wherein the medial cushion base absorbs forces and impacts experienced by the medial articular pad and the lateral cushion base absorbs forces and impacts experienced by the lateral articular pad; and
wherein when the medial and lateral articular pads pivot in a posterior direction, the first and second posterior ends of the medial and lateral articular pads also pivot downwardly towards the first and second downwardly sloped portions.

15. The tibial component of claim 14, wherein the implant tray includes a depressed portion along a posterior edge of one or both of the medial and lateral tray sockets.

16. The tibial component of claim 14, wherein the implant tray includes a depressed portion along a posterior edge of the lateral tray socket.

17. The tibial component of claim 14 further comprising a medial cushion side wall that extends upwardly from the medial cushion base and a lateral cushion side wall that extends upwardly from the lateral cushion base, wherein the medial tray socket includes a first socket side surface and the lateral tray socket includes a second socket side surface, and wherein the medial cushion side wall lines the first socket side surface and the lateral cushion side wall lines the second socket side surface.

18. The tibial component of claim 14 further comprising a first and a second pivot bearing and a first and a second cushion member, wherein the medial articular pad is pivotably supported within the medial tray socket by the first pivot bearing which engages the first socket bottom surface and the first cushion member which is sandwiched between the first pivot bearing and the medial articular pad, and wherein the lateral articular pad is pivotably supported within the lateral tray socket by the second pivot bearing which engages the second socket bottom surface and the second cushion member which is sandwiched between the second pivot bearing and the lateral articular pad.

19. The tibial component of claim 17, wherein the implant tray includes a depressed portion along a posterior edge of the lateral tray socket, and wherein when the lateral articular pad pivots in the posterior direction, the second posterior end pivots towards the depressed portion.
